SEQUENCE LISTING (5 sequences)

SEQ ID NO: 1 - DNA, 1093 bp, Populus tremula x tremuloides
atccctcctattaaatccatctaattgtatggaaaggatggatttataaaattacataggtttrcattgataatatttccaatatttaaaattaaatggtcatcatcattcgattaattcacaatatttagtcctgtctaatcatagaaatcttgtctccgtacatgaaagttgattaaaaataaataagttattaatcttattagttaatttgatgtattgattcatgagtttagtagatttatccatcaaatctaatatgtatttttttagtttttggttcattgatttttctaattgtttttttattttactatttttcagtattgrattgtttaaaaaataaaatttatagtttattttttttttctatagagctatcactatctcaataaatatctttttagagttggtgtttgattttgtaaacatcaaattttatcataaaattaaataaaaataatttataaaaacaacaaagtttaaacctattgaaatctataacccaagtcacaaagttaccaaaacttatttaatttggtatcatctcaataataattaaaaaattattgttttaatttttttttaaatctatcatattttaactaattatttaaattatctttgaacttattaaattaaattrttcatgcttgttagagaaaatattctactaataaataacttttgtcatactaagtcatgaaaaatgtgatctttaatgatacaagaacagaagtccaaaagcagggggggtaaagaaagggagaggttgcctatatatttgccttacacttactctacaaggaaccttttgcacctaatgagcgtaataaatagtatagcaygatagcaatagcacaatagctcagaaatgatatagcttctaactaatatagaagacaacacgagcccaagatgttagccctccaacttatgccaccctaacctgcgtttatactctaaaaagataaatgtagaaaaaaagtgaaaacctaactgtacatggctttagctttactcctactcaaacagccataaaaaatcccaaaacgaggcctctaaaagtgtcctcatcattctgttgcttcag

SEQ ID NO: 2 - DNA, 1245 bp, Populus tremula x tremuloides
gaattcataataattggattggacttgtctattttctttagagatttatcaattgtgttttattttttaacatttattaaattaaatggactaggactatacataccttatctatgatgtatttagtattgcgtttcaaataggggttgaataaattttgaattttatttctgtttaaaattattttttttatgttttcaaatcgttttgatgtgctgatatcaaaaataattttgaaaaaaatatatattattttgatgtatttttgaataaaaaatactttgaaaaacaactggttattacagtctcaaacactcctagtagtcttaggcatagtttaaaactcagtccgacctggcgggtcgacttgggactggaaccgggccgagttaaagaaaaaaataaaaaaagaaaaaacttggtgtgacctggtgactcggttgacccagatgcaacccattgatttttgttttttttaaactaaaattacattgttttgcttttttaaaaaaaaaaattgacccgtcaaacctggtcaaaacccggaatcgaggtcttggaccgggacgggtctaaaaactatgatctttggtcaatatattaagacaacaaggtcaaattaaagggtaatcaaaggagagtttttttttggttggttggttggttttggacctaatcatacaatcataaaataatcatggatcaaaatgtaggtaattcaatcatggtgtgtaatagattttttatatgatcatgtgtcaccagctaggtctttttccaaaattctaacaagaggttggtatatgtaataaaattatctttaaacagacacaacccacaccctctataattaatcttaacaacaagttaatataaaacaattttttttttttgtctaaatatattttgctgaygggtagcatcccaccaactatctggtgcagatagcaactcaagtccttcccaataaacacatttttccatgattcctttcacttcaagtgacaactaaacgaagcaaagcctcgtcaaattaaaaaaaaaaaaaaaagggttccgaataatcataatgtggcttttgagtttgccagtacctgtcatcgaccrgttagcttcaacctccgcaaaaaaacatacagcttcgctataaaacaactccttgtttattaattgttgtcttccacrtctctcaatctctctcaaagaatccctttctttttcctcaaaactcaggggtcagtatct

SEQ ID NO: 3 - DNA, 1133 bp, Populus tremula x tremuloides
atcagcgcatcaaaattatctaaaaacattaaaagtatattaatttcaagaaaaaaaataaaaaaatttaaaaaaaatttacaaataaatttaaaattcaaatacaaaacagattgatatagaatttactattaattatacctctataagcagagacataatattctcttttgagaaacaaaatattattataattgatattccaacaaaaaaacaaatcaatctctttattttagaaagtaaaataacttataattgttcataattgtttgtagatggacgtcatgtttgtcgggggtagacgccctcttggttaaaataaaataaaaaaataattttctaatcttatccaaaatttgtaacctctgycaactgaaatatagtattcttagaattgatattttaaatgaaatccaaatgcttagaatttattttagccatgttgatgagaaagtcaagcaccccattaaataaaataaaataaaataaaaaattacaaggtaattatgcacctagaaatttttatttttttaatataataatacccctttctgactggaaaaggctgcgaggttgtcattcacacaacaaatatcagctatttcaggatccatcgcagtactcgcaatcttcacatttagcaaaatcagagcagcgtatgccctgtttttttcacctttttatcgcactaatcctagagacgaacagtgtttattgtttttctcttaatatttgaccttttcacttattygaaaagtcaaatgttaatttccaccgagctttatcctgtaaatagcgtgtttaatgccagctgtaaataagatagatgatcgattttgtatttacattctcttatcttagacggaaagttattaaaaaaataaaataaaatagaaacctacctaagatgtgaactcttggcgtctctagctgtttctgtctctagaacaacctctacgagtatagccgaagcatatcctagcaagcttgaaccatatctactacctccctccccgctatatatatataaaccactcttcttcctagaaaaaaaataaccctcgatctttctttcttgtgcttatgttgattttgtttgattttcatatatcatgagcaatataccaagatct

SEQ ID NO: 4 - DNA, 1807 bp, Populus tremula x tremuloides
  misc_feature 616: n = A,T,C or G
gatgcaaaagctggaatgagaatgcgctaaatgcgaaaagacagagagagcgaataaatcgtgcaaaaaaaggagtggggtgggtaacgggttgagctagaagaagaaaagggacaagtgcactttaggaggggggcaaccagagcgtagatgataatggttcatgtggaaacaacacacatgagcagttggtgagaacttgaatgaaccctaacagcccaaccaaacccggagccacccttaccgaaccaccacttctaaaagtacaccatgcctttttcttgagcytggttgcacagggtgccrggtggggttgtttcgttttgggtaatcatgcgatagtttaaatacctttgcgataatcatatcaatggygacttttaagcacatgttaggtgctcggttcttatctaaacatggacatggcmacaagagttaatgctaaaataatatacgtacatacctgtgaatgaatcgtcgctgtcttctgattatggcttcaaattaatatgcagataaacaagtgtcgatttttcagtgaagattttatgaaagtgcccgttctccttaagattacctgtgaatgaatcatccctgtcaggctgatcctgggtgntttttccccatgrtgttcggaagatataattatataaatgatggaatttacatgaaataagtttcagtacattcttagcagaaaagcaatatcgacgaagacaaatgatgctgtttaagacaaactggggtaatatcaatttactagtaagagatttgkctgcttttcttaattctcaagaaacttycactaaaatgcacagccatgttaaacaatttacgttcaacttaaaaactaaaactgcaggatgggtagctatccaagaatgattaataaacgattttaacaaagaactacgttttttgtaaacttaattttgatgggcatgtagttaacaagtatttgtcattgatcaattcaagagccatgtctgcatcataattgtgggagtggaggaggcttttgttgctagggaaggaatgccttcttagttcatggctttggacttcggacaaggagcgcatagaatggggttaccatttttgaaaaaaattacatttgaaccctccaactattatcatgtatgtttaatctacaatcctcgtccgctagaagaagtttgggttcaaagtcatccgctcaatgtaaaccatggagaggcagggactaattgaaaatagtatgttagttggagggtctgcatgtattatgtccaaacattctcttattattcctgtatcatctctgagaaattcatccgaaaataataaaacaaaatggccttttttaaaaaaagaagctgatgcataggataccaaaagcgccttgtccattaggagcgtcagactttgaaaataagaccaataattccctgtaagctatcatctcatcttttattttgtttgaacttgtagacgtaggctttaagcgttccatgatgttcagtcacatgttgctgtctacttgattatggaatttaattcattcggctcataagaagataaaaggattatgacgttgaagaactctggtcactccttacttacggtcacataaaaacgatgcatctttccccaccaaccatcttcaagtgaacccactttcccttgcattaggtaaggagtatgggttaagtcatcttcatgaaattagtcccctagtggagctaattctactcactccatatttactcattccactatataacgccctcaacgaccatcctcaaagcaacccaaacaccttcttctc

SEQ ID NO: 5 - DNA, 1494 bp, Populus tremula x tremuloides
  misc_feature 337: n = A,T,C or G
tctgcttatgaatggcatggacaacaagctctgcattttcgcccatttgcaccacaaaatcgttgtgtgaagatttttgaagggcaccagcacaactttgaaaagaacttgtgaaaagatagctggtcaccagatggaagcaaggttactagtgcagatcgcatggcttatatatgaatctgtcaatgagtgtgtcttccacctactgaacctatctttggatcatgcagcagcgacaaacagatttatcttggggaaatctgattactattttgtcagtttgcttaaaacgagttgttagtaacagtgaggatattycggggcttgcttgtgagcncccaacagaaaagattaaacaggttgtaatggatactatgttacattttacttaactttctcatatcctgctgacatgtttgcagttcttttttgcttgttggcgatccttttcaactgaagtatgtacgcatgaatcaattagctcaaaatgtatgttcgagcaatatgcctggtgaatgtttgttaagatacggaaaattaagttactgaacttgtactctttgattttgaaacaagtttccgactatgttcattgtcctagcacggtgatattgatacgtcgaattttcaactatttttgtcttgttttcaactatttctgtcttgattttcaaagtctttttgaaaataaaaaacccaaaaatatgttttcagtgtcttgttagctaaatctgttgtacaaccaagcctccccgacttgctttgtgaaaaagaaagatgggagctgggggttttgtgttgattatacaaaaccttacgagataactgatatacaagcaaaaacaccctcatattattgaatctgcgacttctacagttcatttctgaggaagaattaggtgactactttctggactagatatttccgtgttcaatgcgtcgcaacaattgatttgttggctggattatgaacatgataatcacactatagaaagacgacgattatatttcaagctatggacaatgatgttacctgctcgaagatcaatacgcttgaaggccaagatatgatcascaggtatgcaggcagtccagatggcttcttgggatatgagcttcaagattgttcttgaaaagaaccaaataaattgtaaaattctctgggaatatttttttgtaaaattatcagcattgctattttttttttcaaattcatttaaatattgctattgggttagtagttcagcgcaattaacttagctggcagtctggcattagcttcaaatcatatcacaacgttaaaagcagccgttgcaactagtcagcccactagcatttgctggtgaccaatcagatcataaaaggaaaactagccgttggcaaaccctctttcaacggtcatctccctgcttcatgatatatatgcttagatatcccagtatccccttcaacaaattatctcaatcttcaaaacccagtccccagttacagcag

(19) United States
(12) Patent Application Publication
(10) Pub. No.: US 2007/0180580 A1
(43) Pub. Date: Aug. 2, 2007

(54) Tissue specific promoters

(75) Inventor: Magnus Hertzberg, Umea (SE)

Correspondence Address: MORRISON & FOERSTER LLP, 425 MARKET STREET, SAN FRANCISCO, CA, 94105-2482, US

(73) Assignee: Sweetree Technologies AB, Umea (SE)

(21) Appl. No.: 10/554,173

(22) PCT Filed: Apr. 20, 2004
(86) PCT No.: PCT/SE04/00604

§ 371 Date: Aug. 8, 2006

(30) Foreign Application Priority Data
Apr. 28, 2003 (SE) 0301233-3

(51) Int. Cl. A01H 5/00; C07H 21/04; C12N 15/82; C12N 5/04

(52) U.S. Cl. 800/284; 435/419; 435/468; 536/23.2; 800/295; 800/314; 800/317.2; 800/319; 800/320.1; 800/320.3; 800/306

(57) ABSTRACT

Tissue specific promoters (SEQ. ID. No. 1-5) are disclosed, these being preferentially expressed in the xylem tissue of woody plants. These promoters are used e.g. to specifically regulate gene expression in xylem tissue and alter xylem properties in plants. Transgenic plants, exhibiting modified xylem properties can be produced using these promoters.

The present invention relates to the field of gene technology, and in particular promoters driving gene expression during the development of xylem in any plant, and in particular in the cambial region of a woody plant. The invention makes available such promoters and their practical use.

### BACKGROUND OF THE INVENTION

Xylem formation in plants is an important process developmentally, and it is also very important in the production of fibres that are of economic value. Wood formation in trees is an economically extremely important example of this process. It is the result of a series of defined developmental events from the initial cell division, through cell expansion and secondary wall formation ending in a zone of apoptosis. Wood is produced from the vascular cambium which is a lateral meristem undergoing terminal differentiation ending in autolysis of the cell protoplast.

The present inventors have in the studies leading to the present invention used Poplar that is a well-established model for woody perennials. It has a small genome, only about five times larger than Arabidopsis, another frequently used model. Further, Poplar is a species that is used extensively for forward genetics. Poplar and Arabidopsis are also phylogenetically closely related and access to over 100 000 Poplar ESTs combined with the complete sequence of the Arabidopsis genome make the combination of these two models an excellent choice for studies of xylem differentiation.

When and were a gene is active is important for its function. One level of regulation is the control of transcriptional initiation. The transcriptional expression of a gene is controlled by the cis-acting DNA elements located mainly (but not exclusively) upstream (5′) of the coding sequence. When using gene technology and GMOs (Genetically Modified Organisms) as tools in breeding and product development, there are two basic parts: the gene which gives the wanted effect; and the cis-acting DNA regulatory sequence which determines when, where and the level of gene product expressed. Regulation also occurs at levels such as transcript stability, translational-initiation and -progression, protein activity and so forth. There are genes that have promoters driving their expression during different and specific stages of xylem formation. These promoters will be essential in specifically directing the effect on any gene when genetically modifying xylem properties and altering the amount of xylem produced. The number of available promoters is limited, which means that new ones will be of great importance when tuning and fine tuning the expression of genes in the study and manipulation of xylem formation. Modification of specific processes during cell development in higher species is also of great commercial interest, not only when it comes to improving the properties of trees, but also in other plants whose fibres have commercial use. New tools, such as promoters for such modification, are needed.

### PRIOR ART

Fredrik Sterky et al. (Proc. Natl. Acad. Sci. USA, 1998 (95), 13330-13335) and Magnus Hertzberg et aL. (Proc. Natl. Acad. Sci. USA 2001 (98) 14732-14737) have published the results of a large-scale gene discovery program in two poplar species, comprising 5,629 ESTs from the wood forming tissues of Populus tremula L. x tremuloides Michx. and Populus trichocarpa ‘Trichobel.’ These ESTs represented a total of 3,719 unique transcripts for the two cDNA libraries and putative functions could be assigned to 2,245 of these transcripts. The authors state that the EST data presented will be valuable in identifying genes involved in the formation of secondary xylem and phloem in plants, but fail to give clear directions as to how the identification could be performed.

In the prior art (Hertzberg et al.) transcript profiling of cDNA at defined stages of xylogenesis were performed using cDNA microarray analysis of a poplar unigene set of 2995 ESTs.

However, in order to make possible the practical use of the information published by Sterky et al., and Hertzberg et al., a new approach is needed. U.S. 2002138870 A1 relates to a method of simultaneous transformation of plants with multiple genes from the phenylpropanoid pathways including 4CL, Cald5H, AldOMT, SAD and CAD genes and combinations thereof to produce various lines of transgenic plants displaying altered agronomic traits. The agronomic traits of the plants are regulated by the orientation of the specific genes and the selected gene combinations, which are incorporated into the plant genome. U.S. 2002138870 mentions the desirability of using tissue specific promoters, e.g. such that would confine the expression of the transgenes in developing xylem.

In general, it still remains to identify new, useful promoters. One particular problem remaining is how to identify the potentially most important genes and their corresponding promoters, and to relate these to specific developmental properties of the cell. Another problem is to clone all relevant cis-acting transcriptional control elements so that the cloned DNA fragment drives transcription in the wanted specific expression pattern.

A particular problem is to identify specific promoters, related to specific cell types, developmental stages and/or functions in the plant that are not expressed in other plant tissues.

### SUMMARY OF THE INVENTION

The present invention offers a solution to the above problem by making available isolated nucleic acid sequences, each comprising a promoter sequence specifically expressed in the xylem forming tissue in a plant, said sequence being chosen among SEQ ID NOs 1 through 5; sequences functionally homologous to SEQ ID NOs 1 through 5; and sequences showing at least 90% homology to SEQ ID NOs 1 through 5.

The sequences are presented in the figures, and in the attached sequence listing, prepared using the prescribed software (Patentin 3.1).

Further, the present invention makes available transgenic plants, methods for their production, and seeds and seedlings of such plants, as well as a method for expressing specific genes in the xylem of a plant, and nucleic acid constructs, as defined in the attached claims, incorporated herein by reference.

### SHORT DESCRIPTION OF THE DRAWINGS

The invention will be illustrated in closer detail in the following description, examples and drawings, in which:

FIG. 1 shows consensus sequences from the genome walk fragment. 1a) LMX2 A014P10U; 1b) LMX3 A044P26U; 1c) LMX4 A050P49U; 1d) LMX5 A055P19U; 1e) LMP1 A001P79U, corresponding to the sequences SEQ ID NO 1-5.

Underlined sequence=promoter sequence (part of the GUS construct)

Sequence in italics=sequence from EST

ATG (bold)=Potential translational start codon

FIG. 2 shows a cross section of a hybrid aspen stem, stained with Toulidine blue.

The sample positions are indicated with black bars, below the cross section.

FIG. 3 shows the expression patterns for the 5 ESTs from which the promoters have been cloned. This corresponds to the main expression caused by the respective promoters.

FIG. 4 shows the results from northern blot analysis of expression levels of the selected genes in different plant tissue:

Lane 1: Apical shoot: Top shoot tip of about 1 cm.
Lane 2: Leaf veins: The main veins were collected from old leaves.
Lane 3: Elongating stem: The internodes below the apical shoot, to the eight node of the stem. All leaves and buds were removed.
Lane 4: Old leaves: Healthy leaves more than 15 cm in size. The petiole and main veins were removed.
Lane 5: Phloem: 15 cm stem pieces were collected. These were used for phloem and xylem preparation by scraping the xylem and the inner-side of the bark.
Lane 6: Root: The 2-3 cm apical part of root tips without any root hairs.
Lane 7: Young leaves: Leaves less than 12 cm in size. The petiole and main veins were removed.
Lane 8: Xylem: Tissue collected as for the stem pieces (See Lane 5), the wood side being scraped.

### DESCRIPTION

Definitions

In the description, the term “xylem”, as in xylem forming tissue, is intended to encompass all the vascular tissue through which most of the water and minerals of a plant are conducted. The principle conducting cells of the xylem are the tracheids and the vessel elements, or vessel elements. Both are elongated cells that have secondary walls and lack protoplasts at maturity. Xylem tissue also contains parenchyma cells that store various substances.

The term “vascular cambium” is meant to encompass the vascular meristem mother cells, the division of which produces secondary xylem and secondary phloem.

The terms “wood” or “wood forming cells” are meant to encompass all cells whose metabolic events and processes are involved in the development of the vascular meristem and all of its end products. The term “wood” is also used in its most general sense, encompassing both softwood and hardwood.

The term “wood formation properties” encompasses both biological, chemical and physiological properties, for example but not limited to cell elongation, cell expansion, apoptosis, carbohydrate composition, fibre length, fibre thickness, and other fibre properties.

“Homology” is here used as a measure of the similarity between sequences; the greater the sequence homology between two sequences, the greater degree of hybridisation. Hybrid formation can be measured by a person skilled in the art, using known procedures.

In the present invention, sequences sharing at least 90%, preferably at least 91%, more preferably at least 92%, more preferably at least 93%, more preferably at least 94%, more preferably at least 95%, more preferably at least 96%, more preferably at least 97%, more preferably at least 98%, and most preferably at least 99% homology are encompassed.

“Functionally homologous” means sequences sharing perhaps a lower structural homology with the disclosed sequence, but exhibiting homologous function in vivo, in either the healthy or the diseased organism, e.g. coding the same or highly similar proteins with similar cellular functions. The present invention encompasses sequences functionally homologous to the sequences disclosed.

“Complementary” in the context of this description refers to the capacity for precise pairing between two nucleotides.

Further, in the context of the present invention, “hybridization” refers to hydrogen bonding, which may be Watson-Crick, Hoogsteen or reverse Hoogsteen hydrogen bonding, between complementary nucleoside or nucleotide bases. Thus complementarity and hybridisation are terms used to indicate a sufficient degree of complementarity or precise pairing such that stable and specific binding occurs between the oligonucleotide and the DNA or RNA target.

An antisense compound is specifically hybridisable when binding of the compound to the target DNA or RNA molecule interferes with the normal function of the target DNA or RNA to cause a loss of utility, and there is a sufficient degree of complementarity to avoid non-specific binding of the antisense compound to non-specific target sequences under conditions in which specific binding is desired.

The phrase “hybridisation under stringent conditions” refers to criteria regarding temperature and buffers well known to those skilled in the art. See e.g. Sambrook, J., Fritsch, E. F. and Maniatis, T., Molecular cloning: A laboratory manual, 2nd Edition, Cold Spring Harbour Laboratory Press, USA (1989).

“Functionally inserted” or “operationally inserted” denotes that a sequence has been inserted in a host genome in such orientation, location and with such promoters, where applicable, that the correct expression of said sequence occurs.

“Modulation” as used in this context means either an increase (stimulation) or a decrease (inhibition) in the expression of a gene. In the context of the present invention, increase is the preferred form of modulation of gene expression and mRNA is a preferred target.

### DETAILED DESCRIPTION

The selection of developmental stage specific expressed genes was used to identify promoters that direct gene expression to specific cells within the xylem forming tissue. When cloned and analysed, these promoters were shown to be tissue specific. Being in possession of these promoters, it becomes possible to drive the expression of any gene of interest in specific stages of xylem development and effect the production and properties of the xylem (wood). The methods to alter expression include ectopic and over-expression of the gene of interest, antisense regulation, RNA interference, gene silencing, the use of ribozymes etc.

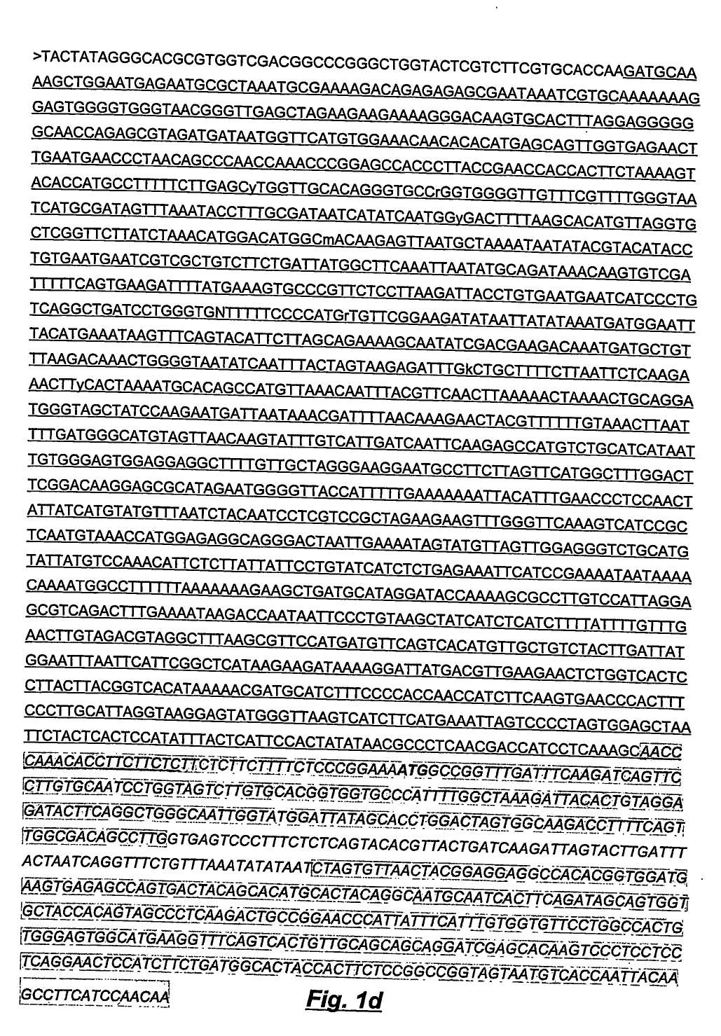

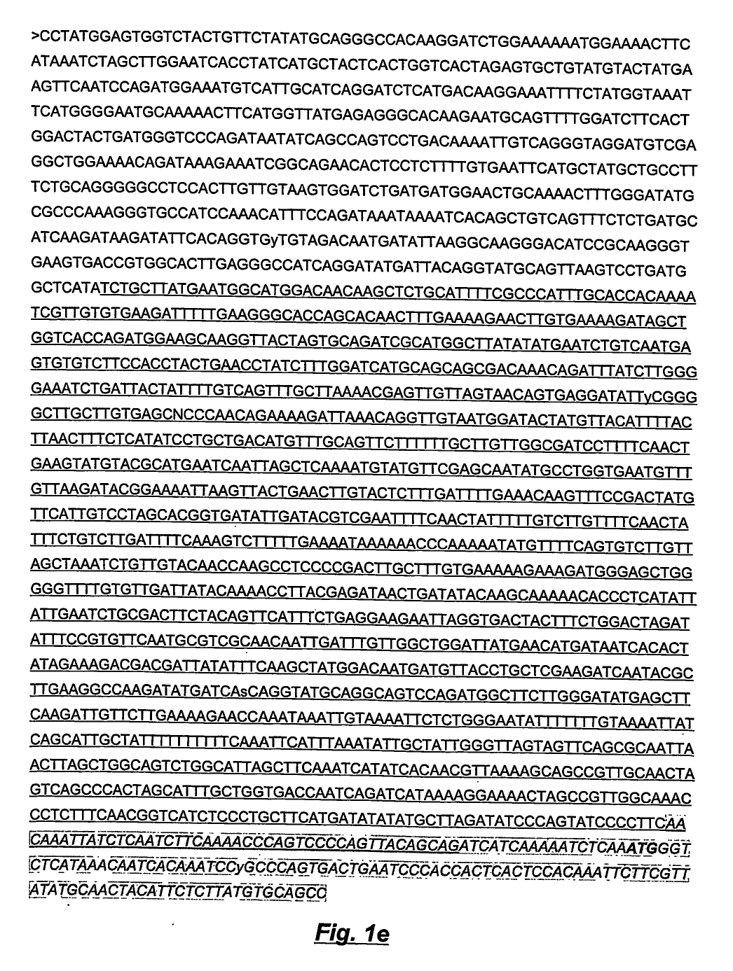

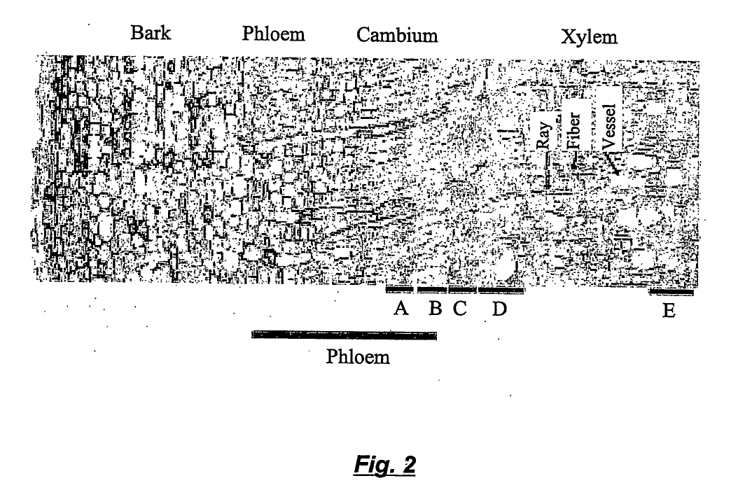

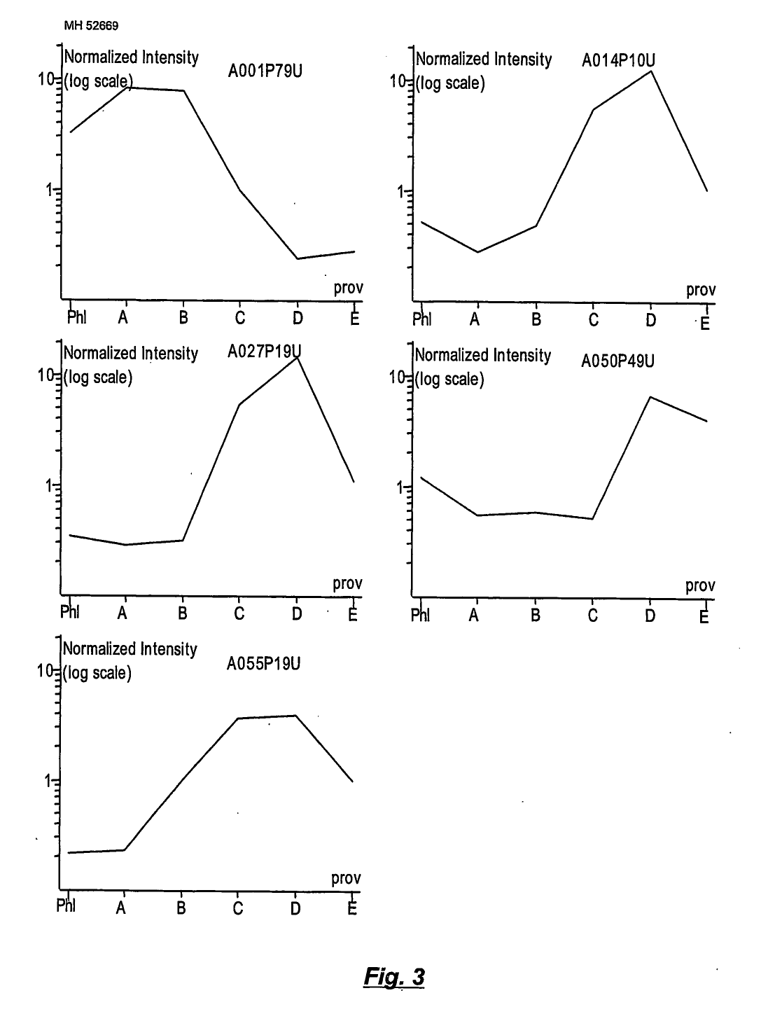

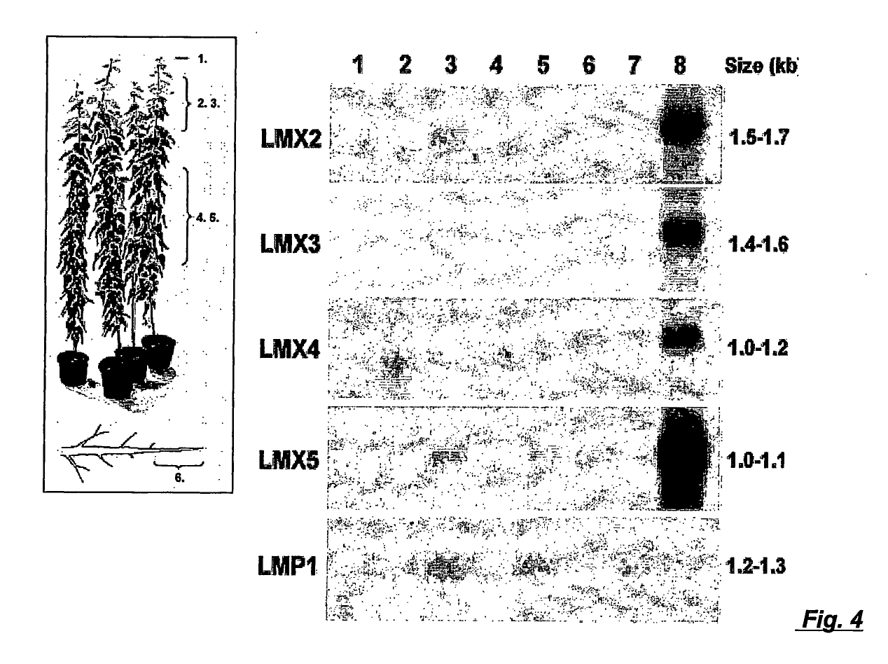

Libraries were created from regions within the xylem forming tissue, representing the areas of cell division (A), cell expansion (ABC); secondary cell wall formation (CDE); cell apoptosis (E). See FIG. 2.

Tissue samples were prepared by taking tangential sections from the cambial region of the stem. Transverse hand-sections were collected in parallel to allow later identification of the exact location and tissue content of each sample. The individual sections measured 30 μm×2 mm×20 mm corresponding to a fresh weight of approximately 0.5 mg. Sections were pooled together into different developmental zones as indicated in FIG. 2. The selection of different zones was based on the radial diameter and anatomical features of the cells. Within these zones, the gene profiles showed a very high degree of tissue specificity.

The present invention discloses gene promoters and their use, said promoters identified via the characterisation of cDNA microarray analysis, selected from a high number of sequences, found in undifferentiated meristematic cells in the cambium through to matured xylem elements ending in cells entering programmed cell death or apoptosis.

The present invention makes available isolated nucleic acid sequences comprising a promoter sequence specifically expressed in the xylem forming tissue in a plant, wherein said sequence is chosen among: SEQ ID NOs 1 through 5; sequences comprising one of said sequences SEQ ID NOs 1 through 5; sequences being functionally homologous to SEQ ID NOs 1 through 5; or sequences showing at least 90% homology to one of SEQ ID NOs 1 through 5, preferably at least 91%, more preferably at least 92%, more preferably at least 93%, more preferably at least 94%, more preferably at least 95%, more preferably at least 96%, more preferably at least 97%, more preferably at least 98%, and most preferably at least 99% homology.

The above definitions do not exclude each other, but may act together. For example a sequence exhibiting lower homology, e.g. 80% homology or 70% homology, but being functionally equivalent to these sequences, is included in this definition. The invention also encompasses sequences capable of hybridising under stringent conditions to at least one of these sequences.

One aspect of the invention is nucleib acid sequences as defined above, wherein the promoter sequence is expressed in a higher plant. Preferably said plant is a woody dicotyledon. Examples of plants include but are not limited to hardwood species, such as poplar, aspen, birch, willow, eucalyptus, sweetgum (liquidamber) etc., and softwood species (conifers) spruce, larch, hemlock and pine etc. Other plants of interest are so called fibrous plants, for example, but not limited to cofton, hemp, sisal, flax, etc. The promoter could be used in other economically significant plants like wheat, maize, potatoes, oil seed rape etc in order to drive the expression of genes during vascular development.

The present invention makes available transgenic plants exhibiting modified properties when compared to the wild-type of said plant, e.g. modified wood formation properties, modified apoptosis properties, altered rooting properties, altered flowering or leaf pattern etc in comparison to the wild-type of said plant, wherein at least one of the above sequences is functionally inserted into said transgenic plant.

The transgenic plant is preferably a woody plant, most preferably a woody dicotyledon. Examples of transgenic plants include but are not limited to hardwood species, such as poplar, aspen, birch, willow, eucalyptus, sweetgum (liquidamber) etc., and softwood species (conifers) spruce, larch, hemlock and pine etc. Other plants of interest are so called fibrous plants, for example, but not limited to cotton, hemp, sisal, flax, etc. The promoter could be used in other economically significant plants like wheat, maize, potatoes, oil seed rape etc in order to drive the expression of genes during vascular development.

The present invention also makes available a method for expressing specific genes in a specific zone of the plant, preferably the xylem of a plant, wherein at least one of the above sequences is used, functionally inserted into the plant. Consequently, the present invention makes it possible to regulate the xylem forming tissue development process in a plant, and in particular woody plants, wherein a promoter as defined above is used.

The invention also encompasses plant reproduction products of the plants of the invention, carrying the sequences in their genome, e.g. seeds, fruits, cuttings and parts of the plants, such as protoplasts, plant cells, calli or roots.

An intermediate, also encompassed by the invention, is a nucleic acid construct comprising a sequence as defined above. Such construct preferably comprises a vector chosen among a plasmid, a cosmid, a virus or a bacteriophage.

The present invention also includes a method for the production of a transgenic plant exhibiting modified wood formation and/or apoptosis properties compared to the wild type of said plant, wherein a promoter as defined above is functionally inserted in the plant.

Those skilled in the art are well able to construct vectors and design protocols for recombinant gene expression.

Suitable vectors can be chosen or constructed, containing appropriate regulatory sequences, including promoter sequences, translational leader sequences, terminator fragments, polyadenylation sequences, enhancer sequences, effecter genes, marker genes and other sequences as appropriate. For further details see, for example, Molecular Cloning: a Laboratory Manual: 2^{nd} edition, Sambrook et al., 1989, Cold Spring Harbor Laboratory Press.

Many known techniques and protocols for manipulation of nucleic acids, for example in preparation of nucleic acid constructs, mutagenesis, sequencing, introduction of DNA into cells and gene expression, and analysis of proteins, are described in detail in Ausabel et al. (Eds.), Current Protocols in Molecular Biology, 2^{nd} edition, John Wiley & Sons, 1992.

Selectable genetic markers may be used consisting of chimaeric genes that confer selectable phenotypes such as resistance to antibiotics. Such selectable markers can include kanamycin, hygromycin, phosphoinotricin, chlorsulfron, methotrexate, gentamycin, spectinomycin, imidazolinones, d-aminoacids and glyphosate.

Nucleic acid molecules and vectors according to the present invention may be provided isolated and/or purified from their natural environment, in substantially pure or homogeneous form, or free of nucleic acid or genes of the species of interest or origin other than the sequence encoding promoter with the required function. Nucleic acid according to the present invention may include genomic DNA and may be wholly or partially synthetic. The term “isolate” encompasses all these possibilities.

Plants transformed with these DNA segments containing at least one of the sequences SEQ ID NO 1-5, a functionally homologous sequence to one of thesaid sequences, or a sequence exhibiting a homology of at least 90% to one of said sequences, may be produced by standard techniques which are already known for the genetic manipulation of plants. DNA can be transformed into plant cells using any suitable technology, such as a disarmed Ti-plasmid vector carried by Agrobacterium exploiting its natural gene transfer ability, particle or microprojectile bombardment, microinjection, electroporation, other forms of direct DNA uptake, liposome mediated

DNA uptake. Physical methods for transformation of plant cells are reviewed in Oard, 1991, Biotech. Adv. 9: 1-11.

Following transformation, a plant may be regenerated, e.g. from single cells, callus tissue, leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II, and III, Laboratory Procedures and Their Applications, Academic Press, 1984; Weissbach, Methods for Plant Molecular Biology, Academic Press, 1989; or Clapham et al., Gene Transfer by particle bombardment and Embryonic cultures of Picea abies and the production of transgenic plantlets. Scandinavian Journal of Forest research 15 (2000) 151-160.

The particular choice of a transformation technology will be determined by its efficiency to transform certain plant species as well as the experience and preference of the person practicing the invention with a particular methodology of choice. It will be apparent to the skilled person that the particular choice of a transformation system to introduce nucleic acid into the plant cells is not essential to or a limitation of the invention, nor is the choice of technique for plant regeneration, or the technique chosen for the later multiplication of the transformed plants.

### EXAMPLES

1. The Experimental System

Xylem development is a specialised form of plant development that has been studied in several systems including Arabidopsis and Zinnia. However, for in vivo studies the Poplar system is superior because of the highly organised structure of the Poplar stem allowing precise and accurate sampling of specific tissues at defined developmental stages.

Secondary xylem is highly organised, and the development of different cell lines are strictly coordinated in space. The present inventors have separated different tissues at defined developmental stages from the cambial region using a cryotom, producing by tangential cryosectioning 30 μm sections, with a fresh weight of about 0.5 mg. This dissection technique gives samples highly enriched for cells at specific stages of differentiation as shown in FIG. 2. Five samples were collected to encompass the ontogeny of wood formation: A) meristematic cells, B) early expansion, C) late expansion, D) early secondary wall formation and E) late stage of cell maturation.

Like other meristems, the main function of the vascular cambium (FIG. 2, zone A) is cell division and setting out patterns for differentiation. But unlike apical meristems, the vascular cambium contains two morphologically distinct initials; axially elongated fusiform initials and isodiametrical ray initials. Both types of initials divide periclinally to give rise to radial cell lines of phloem and xylem elements. Derivatives from the fusiform initials make up the axial cell system. In the wood of Angiosperm trees such as poplar this consists of two major cell types, vessel elements that have a function in water transport and fibres that give mechanical support to the stem. The ray initials give rise to the horizontally oriented ray cells that have a major function in lateral transport of carbohydrates supplied by the phloem sap and minerals supplied in the xylem sap. As the stem increases in diameter, more initials are required. Fusiform initials are formed by pseudotransverse anticlinal divisions, which result in a shortening of the daughter cells. Therefore fusiform initials both divide and elongate in the cambial meristem.

In the cell expansion zone, cambial derivatives enlarge with different polarities depending on cell type (FIG. 2, zones B and C). Developing fibres expand radially and increase in length by tip growth, whereas vessels expand only in the radial direction. Vessel expansion is rapid and much more extensive than fibre expansion, giving these cells their morphological characteristics. Vessel differentiation also progresses at a much faster rate than the development of fibres and rays, which are synchronised. Vessels have thereby formed secondary walls already in zone C and completed their maturation in zone E. During the stages of division and expansion (zones A, B and C) there is a requirement for a continuous biosynthesis of primary walls, which in poplar mainly consist of cellulose, hemicellulose and pectin. Fibre elements and ray cells in contact with vessels (contact rays) differentiate slightly faster than fibres and ray cells without contact to vessels (isolation rays). Therefore, zone C contains a mixture of primary and primary+secondary walled cell types.

As soon as cell expansion is complete, the secondary wall is deposited in all xylem cells (zone D). One of the characteristic features of this stage of development is that the random organisation of cellulose microfibrils present in the primary wall now changes to a highly organised helical structure in the secondary cell wall. The cellulose and hemicellulose framework of secondary walls is accumulating in several wall layers (S1, S2, S3) that can be recognised from the degree of cellulose microfibril orientation. At the final stage of secondary wall thickening the interspace within the cellulose and hemicellulose network is lignified, starting from the cell corners and progressing inwards. During the formation of the secondary wall, the xylem elements are also extensively sculptured to form the network of pits and pores, which allow for vessel/vessel and vessel/ray contacts. This activity involves patterned degradation of the primary wall. At the end of the maturation process (zone E), fibres enter programmed cell death, (vessels enters into programmed cell death earlier in zone C and D). In Contrast, ray cells continue to contribute to the lignification of the surrounding cells and remain alive in the sapwood for several years.

2. Promoter Cloning and Sequencing

The present inventors have approached the problem of identifying tissue specific gene promoters by performing tissue specific transcript profiling. With cryo-sectioning it becomes possible to obtain samples that are highly enriched with specific types of cells. The organised nature of the tissue in connection with the cryo-sectioning technique provides samples that are highly enriched in specific cell types and thus enabled us to perform tissue specific transcript profiling.

The present inventors cloned the 5′ upstream sequences of 5 genes in Hybrid aspen (Populus tremula x tremuloides). These genes are all expressed during specific phases of xylem (wood) formation as based on the cDNA microarray analysis (Hertzberg et al., 2001 PNAS). A number of selection criterias were set up to ensure the wanted result. The selection criteria included: the EST had to have a BLASTX hit in the Arabidopsis proteome indicating that the EST contained the full open reading frame (ORF), and thereby probably contains the full transcript; the EST had to have an expression pattern, based on cDNA microarray data (Hertzberg et al) that showed a clear differential expression in the regions of interest, and also had an strong hybridization signal suggesting that the promoter driving the expression of that EST is strong.

The five genes I-V are represented by the EST's given in Table 1 below. In the table, the name used for the cloned promoter fragment is also indicated, as well as the zone where the promoter is predominantly active.

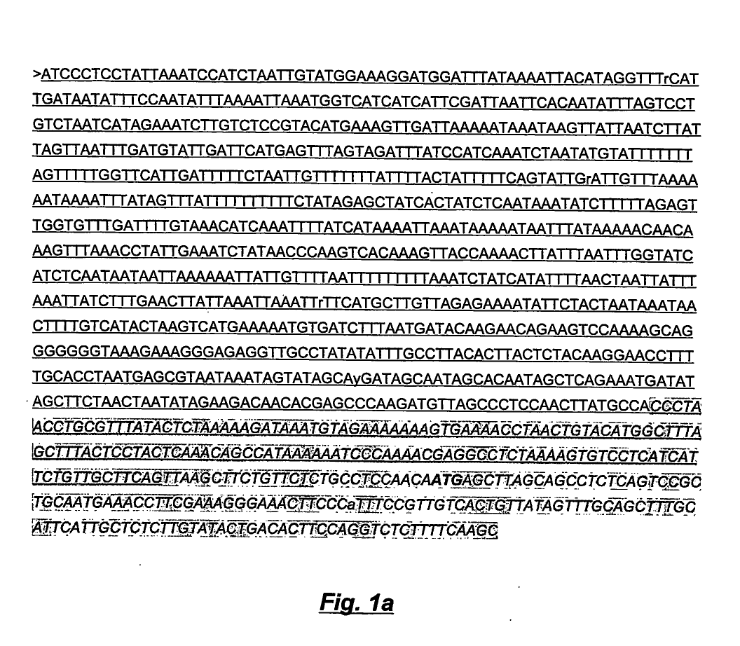

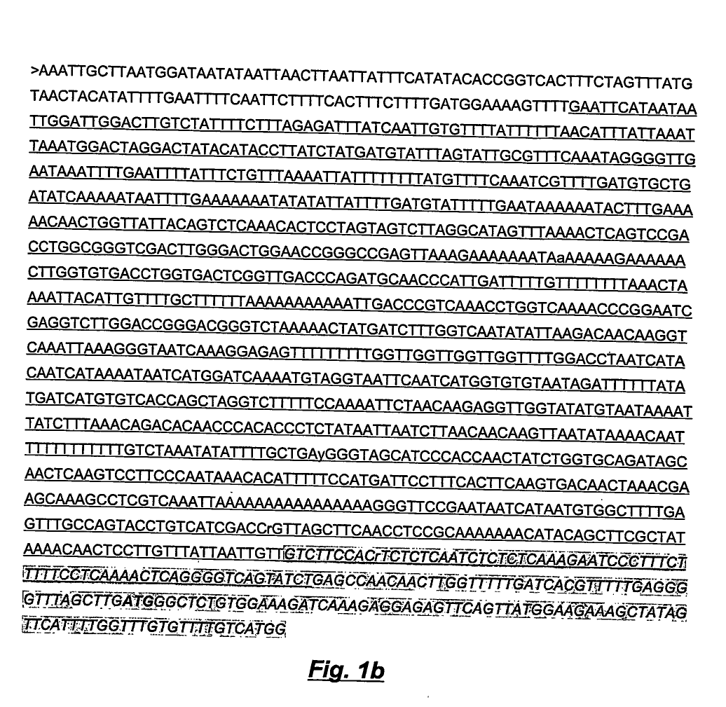

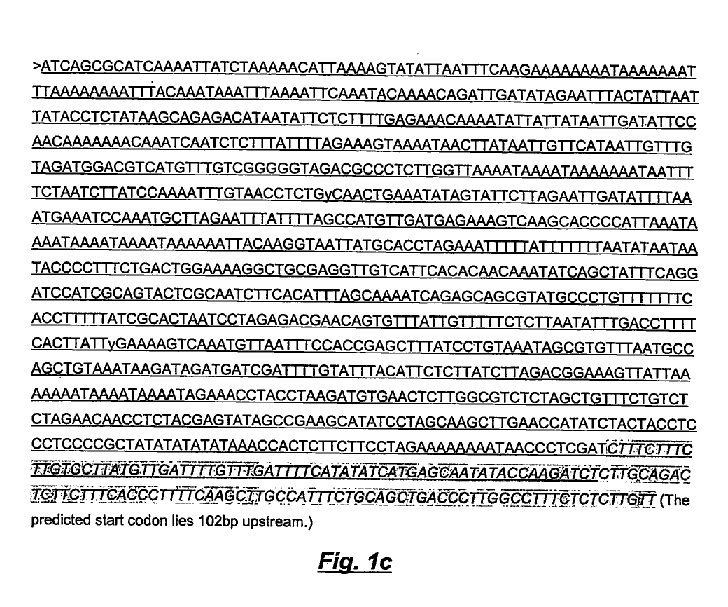

TABLE 1

| | | | Name of | Gene |
|---|---|---|---|---|
| GeneBank | Internal | Expressed in | cloned | number in |
| EST nr | EST nr: | zon: | promoter | |
| Al162215 | A014P10U | C-D | LMX 2 | 1 (FIG. 1a) |
| Al163548 | A044P26U | C-D | LMX 3 | 2 (FIG. 1b) |
| Al163880 | A050P49U | D-E | LMX 4 | 3 (FIG. 1c) |
| Al164126 | A055P19U | C-D | LMX 5 | 4 (FIG. 1d) |
| Al161513 | A001P79U | A-B | LMP 1 | 5 (Fig. 1e) |

The expression pattern for the Al163548 EST is based on the expression pattern of Al162928 (A027P19U) which is an EST originating from the same gene.

The expression pattern for these genes in different parts of the plant were analysed with northern blot analysis. This data shows that the genes LMX2 through LMX5 all are predominantly expressed in secondary xylem. The LMP1 gene has a more general expression pattern but it has the highest signal in samples containing vascular cambium (3 and 5) as would be suggested from the microarray analysis.

These results indicate that these genes are predominantly expressed during cambial cell differentiation and not in other processes, which was a prerequisite for going further and cloning the 5′ regions (promoters) of these genes.

Northern Blot Analysis, Material and Methods

Total RNA was prepared using a CTAB based method (Plant Molecular Biology Reporter volume 11 (2) 1993) and the RNA was subsequently cleaned by using the QIAGEN Rneas® Plant Mini Kit. RNA concentrations were determined using Ribo Gree® RNA quantification kit (Molecular Probes Eugen, Oregon) and the quality of the RNA was checked by agarose gel analysis. Equal amounts of RNA (15 μg) from the different samples were separated on a formaldehyde agarose gel (Sambrook, J; Fritsch, E, Maniatis, T., Molecular cloning: A laboratory manual, Cold Spring Harbour Press, New York, 1989). The RNA was then blotted to a Hybond-N+ filter according to standard methods and UV-crosslinked to the filter. EcoR1/Not1 fragments were recovered from the EST clones. The fragments were labelled using the Strip-EZ™ DNA labelling kit from Ambion. Hybridizations were performed in Church buffer at 65° C. (Church, G.M. and Gilbert, W. Genomic Sequencing, Proc. Natl. Acad. Sci. USA 81. 1991-1995, 1984). The filters were then washed (at 65° C.) twice for 15 min in 0.5% BSA, 1 mM EDTA, 40 mM NaHPO₄ pH 7.2, 5% SDS and four times for 5 min in 1 mM, EDTA, 40mM NaHPO₄ pH 7.2, 1% SDS. The hybridization results were analysed using a phosphoimager (Molecular Imager System GS525). Each filter were used up to two times and stripped using the Strip-EZ™ DNA kit protocol before re-probing.

Promoter Cloning and Plant Transformation

Upstream genomic DNA sequences were cloned using the Universal GenomWalker™ kit from Clontech according to the manufactures recommendations. 8 genome walker libraries were constructed and used. The longest or in some cases the most specific amplified fragment was cloned into pGEM-T-easy vector (Promega) and sequenced. After sequencing the EST clone and a presumed translational start codon (based on the start codon in the best blastx hit in the Arabidopsis proteome) were mapped on to the sequence (FIG. 1, see sequences 1-5). Based on this, the presumed promoter was cloned from hybrid aspen genomic DNA using PCR and subsequently ligated into pPCV812.km (R. Walden, C. Koncz, J. Schell. Methods in Plant Moll Cell Biol. 1, 175 (1990)) or into pGWFS7 (M. Karimi, D. Inze and A. Depicker. Trends in Plant Sciences 7 (5)193-195, 2002) producing a transcriptional promoter GUS reporter construct. The promoter fragment was designed from 10-50 bp upstream the start codon to as far 5′ as possible, giving 5′ sequences ranging from 1093 to 1807 bp. These constructs were transformed into hybrid aspen clone T89 using agrobacterium and transgenic plants were generated (Nilsson O. et al., Spatial pattern of cauliflower mosaic virus 25S promoter luciferase expression in transgenic hybrid aspen trees monitored by enzymatic assay and non-destructive imaging. Transgenic research 1. 209-220 (1992)). Following root initiation the plants were potted in 50% expanded clay (Leca attklinker, size 2-6 mm) and 50% soil (Weibulls kronmull, YrkesPlantJord) and grown in green house with 18 h light and 6 h dark periods using Osram Powerstar HQI BT 400 W lamps as supplementary light.

GUS Analysis

To visualise the expression of the GUS reporter gene, tissue section and parts were stained in X-gluc essentially according to Nilsson et al (The Agrobacterium rhizogenes rolB and roIC promoters, Physiologia Plantarum 100: 456462 (1997)) and to Regan et al. (Accurate and high resolution in situ hybridization analysis of gene expression in secondary stem tissues, Plant J. 19, 363-369 (1999)). Initial results on the promoter LMX5 show vascular associated expression (both in Arabidopsis and in hybrid aspen) and results for LMP1 showed an vascular associated expression in Arabidopsis and an root- and apical-meristem associated expression in hybrid aspen during plant regeneration.

These results indicate that the cis acting elements necessary for xylem associated expression were included in these constructs, this is not obvious and is therefore a surprising result, when the localisation of these sequences could be both further upstream (5′) and also 3′ of the transcription start.

A further and more detailed analysis of the Lmp1 and Lmx5 promoters in larger 2-3 m high plants, performed during the priority year, revealed highly specific expression patterns for these two promoters respectively. In the stem of the Lmp1 Gus lines the Gus activity were mainly present in the cambium cells +the expanding developing xylem but also to a lesser extent in the young cells producing 2^{nd} cell walls. Lmx5 had the opposite expression pattern with the highest expression in young cells producing secondary cell walls and a lower expression in the cambium and expanding xylem cells. Both Lmp1 and Lmx5 are expressed in all the cells of the cambium and young secondary cell wall forming cells.

Both these promoters were active in the rot tip and in the vasculature of leafs and roots. None of these promoters seem to have any expression in the apical meristem but they both have expression in young xylem, especially Lmp1 which is expressed close to the apical meristem in the developing vasculature. Lmx5 is also expressed in the developing vasculature but at an later stage.

Lmx2, Lmx3 and Lmx4 were analysed in Poplar transgenics in late stages of tissue culture. These three promoters were cloned into the pKGWFS7 vector. Lmx2 was expressed in vasculature of leafs stems and roots. It was also expressed in the root tip. In the stem it is seen that the main expression is in the cambium and all the way into the 2nd cell wall forming cells. Lmx3 has an similar expression pattern with the notable difference that it is not expressed in the root tip but clearly in the vasculature of the root from about 0.5 to about 7 mm from the root tip. Lmx3 is also expressed in the apical meristem and young leaf primordia and are later in leaf and primary stem development confined to the vasculature. The Lmx4 gene was lowly expressed in the young tissues present in tissue culture, however an expression in the vasculature can clearly be seen, especially in the oldest vasculature present in the analysed samples.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

### REFERENCES

Ausabel et al. (Eds.), Current Protocols in Molecular Biology, 2^{nd} edition, John Wiley & Sons, 1992
Church, G. M. and Gilbert, W. Genomic Sequencing, Proc. Natl. Acad. Sci. USA 81. 1991-1995,1984
Clapham et al 2000, Gene Transfer by particle bombardment and Embryonic cultures of Picea abies and the production of transgenic plantlets. Scandinavian Journal of Forest research 15,151-160
Hertzberg, M. et al., Proc. Natl. Acad. Sci. USA 2001 (98) 14732-14737
Karimi, M., Inze D. and Depicker, A., Trends in Plant Sciences 7 (5)193-195, 2002
Nilsson O. et al., Spatial pattern of cauliflower mosaic virus 25S promoter luciferase expression in transgenic hybrid aspen trees monitored by enzymatic assay and non-destructive imaging. Transgenic research 1. 209-220 (1992)
Nilsson et al., The Agrobacterium rhizogenes roIB and roIC promoters, Physiologia Plantarum 100: 456462 (1997)
Oard, 1991, Biotech. Adv. 9: 1-11
Regan et al., Accurate and high resolution in situ hybridization analysis of gene expression in secondary stem tissues, Plant J. 19, 363-369 (1999) Sambrook, J., Fritsch, E. F. and Maniatis, T., Molecular cloning: A laboratory manual, 2nd Edition, Cold Spring Harbour Laboratory Press, USA (1989)
Sterky, F. et al., Proc. Natl. Acad. Sci. USA, 1998 (95), 13330-13335
Vasil et al., Cell Culture and Somatic Cell Genetics of Plants, Vol I, II, and III, Laboratory Procedures and Their Applications, Academic Press, 1984
Walden, R., Koncz, C., Schell, J., Methods in Plant Moll Cell Biol. 1, 175 (1990)
Weissbach, Methods for Plant Molecular Biology, Academic Press, 1989

1-17. (canceled)

18. An isolated nucleic acid sequence comprising a promoter sequence specifically expressed or active in the xylem forming tissue of a plant, wherein said promoter sequence is selected from the group consisting of: SEQ ID NO:1, SEQ ID NO:2, SEQ ID NO:3, SEQ ID NO:4, SEQ ID NO:5, a sequence having at least 90% identity to SEQ ID NO:1, a sequence having at least 90% identity to SEQ ID NO:2, a sequence having at least 90% identity to SEQ ID NO:3, a sequence having at least 90% identity to SEQ ID NO:4, and a sequence having at least 90% identity to SEQ ID NO:5.

19. A genetic construct comprising the promoter sequence of claim 18.

20. The genetic construct of claim 19, wherein the construct comprises a vector.

21. The genetic construct of claim 20 wherein the vector is selected from the group consisting of a plasmid, a cosmid, a virus and a bacteriophage.

22. A transgenic plant comprising the genetic construct of claim 20.

23. The transgenic plant of claim 22 wherein said plant is a woody plant or a fibrous plant.

24. The transgenic plant of claim 22 wherein the transgenic plant is selected from the group consisting of poplar, aspen, birch, willow, eucalyptus, sweetgum (liquidamber), spruce, larch, hemlock, pine, cotton, hemp, sisal, flax, wheat, maize, potatoes, and oil seed rape.

25. The transgenic plant of claim 22 wherein said transgenic plant exhibits modified wood formation properties compared to the wood formation properties of a plant lacking said genetic construct.

26. The transgenic plant of claim 22 wherein said transgenic plant exhibits modified apoptosis properties compared to the apoptosis properties of a plant lacking said genetic construct.

27. A method for expressing a nucleotide sequence in the xylem of a plant, comprising coupling the promoter sequence of claim 18 to a nucleotide sequence to form a genetic construct, transforming said plant with said genetic construct and expressing said nucleotide sequence in the xylem of said plant.

28. Propagating material of the transgenic plant of claim 22.

29. The propagating material of claim 28 wherein said propagating material is selected from the group consisting of seeds, fruits, cuttings and parts of said plants.

30. The propagating material according to claim 28 wherein said parts of said plants are selected from the group consisting of protoplasts, plant cells, calli and roots.
